(19) United States
(12) Patent Application Publication
(10) Pub. No.: US 2026/0314918 A1
(43) Pub. Date: Oct. 15, 2026

(54) VISUALIZATION LARYNGOSCOPE AND METHODS OF MAKING AND USING SAME

(71) Applicant: Welsh Innovation, LLC, Newtown, PA (US)

(72) Inventor: Paul C. Frake, Newtown, PA (US)

(73) Assignee: Welsh Innovation, LLC, Newtown, PA (US)

(21) Appl. No.: 19/175,089

(22) Filed: Apr. 10, 2025

(51) Int. Cl. A61B 1/267; A61B 1/00

(52) U.S. Cl. CPC A61B 1/267; A61B 1/00071

(57) ABSTRACT

A laryngoscope includes a handle and a blade coupled to the handle, the blade having a proximal section and an intermediate section, the proximal section having only an upper member and a first side member, and the intermediate section having the upper member, the first side member and a lower member.

### CROSS-REFERENCE TO RELATED APPLICATIONS

The present application is a continuation of International Application No. PCT/US23/36226, filed Oct. 30, 2023, which claims priority to U.S. Provisional Ser. No. 63/382,710, filed Nov. 7, 2022, the content of which is hereby incorporated in its entirety as if fully set forth herein.

### FIELD OF THE DISCLOSURE

The present disclosure relates generally to laryngoscopes. More specifically, the present disclosure relates to laryngoscopes for improved visualization and operative exposure of the pharynx, larynx and/or cricopharyngeus muscle.

### BACKGROUND OF THE DISCLOSURE

Laryngoscopy is a procedure in which an instrument in inserted into the throat in order to visualize relevant anatomy (e.g., the pharynx and/or larynx). Traditionally, operative laryngoscopy has been performed with rigid tubular laryngoscopes of various configurations for over a century. Under ideal circumstances this allows direct, line-of-sight, access to the pharyngeal and laryngeal structures. Further detail can be provided by addition of a surgical microscope or endoscope via this rigid linear laryngoscope.

However, the anatomic structures of the mouth and throat do not generally follow a straight line. Therefore, complications of injury to the associated anatomic structures may result from adaptation of a straight rigid laryngoscope to the patient. This includes mucosal injury, tongue swelling, pain or numbness, dental injury, jaw injury, and/or bleeding. Additionally, an inability to see the anatomic structures of interest may result in an aborted or failed procedure, which wastes valuable time and resources. The latter group of patients may include patients whose anatomy precludes appropriate surgical access using currently available technology.

Once proper visualization has been achieved, surgery on the relevant anatomic structures requires utilization of micro instruments with long handles. The distance between the surgeon and the laryngeal anatomy is approximately 15-40 centimeters. Microsurgery at this distance requires the instruments to be braced or balanced for stability.

In recent years, several groups have tried to adapt current equipment to this anatomic dilemma by creating custom curved rigid tubular laryngoscopes and curved instruments, single handed application of an intubating video laryngoscope, and flexible remote instrumentation (i.e., laparoscopic or robotic). Each of these approaches has its own limitations which include, for example, access to the larynx only, unnatural backhanded surgical positioning or single-handed operation, lack of instrument stabilization, and/or increased cost.

Thus, there exists a need for devices that improve upon and advance the methods of operative laryngoscopy.

### SUMMARY OF THE DISCLOSURE

In some embodiments, a laryngoscope includes a handle and a blade coupled to the handle, the blade having a proximal section and an intermediate section, the proximal section having only an upper member and a first side member, and the intermediate section having the upper member, the first side member and a lower member.

In some embodiments, a laryngoscope includes a handle, and a blade coupled to the handle, the blade having a proximal section and an intermediate section, the proximal section having an upper member and a first side member, and the intermediate section having the upper member and the first side member and defining a window formed in the upper member.

In some embodiments, a surgical method includes providing a laryngoscope including a handle, a blade coupled to the handle, the blade having a proximal section and an intermediate section, the proximal section having only an upper member and a first side member, and the intermediate section having the upper member, the first side member and a lower member, introducing the blade of the laryngoscope to a patient's throat, and passing an instrument through the proximal section of the blade to the intermediate section of the blade.

In some embodiments a surgical method includes providing a laryngoscope including a handle, a blade coupled to the handle, the blade having a proximal section and an intermediate section, the proximal section having only an upper member and a first side member, and the intermediate section having a curved upper jaw and a curved lower jaw, the upper jaw and the lower jaw being joined at opposing junctions, placing a cricopharyngeus muscle between the curved upper jaw and the curved lower jaw, and passing a treatment device through the laryngoscope to treat the cricopharyngeus muscle.

### BRIEF DESCRIPTION OF THE DISCLOSURE

Various embodiments of the presently disclosed laryngoscopes are disclosed herein with reference to the drawings, wherein:

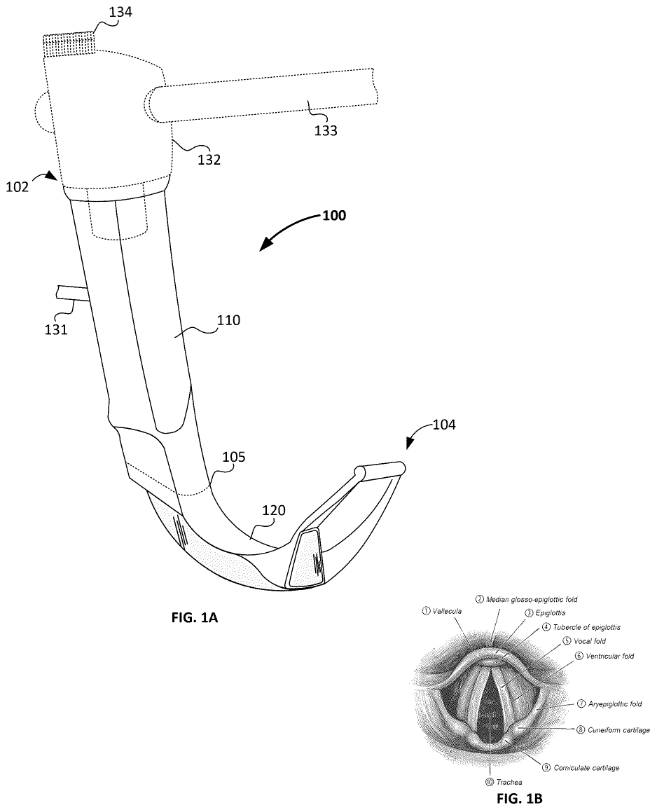

FIG. 1A is a schematic perspective view of a laryngoscope according to one embodiment.

FIG. 1B is a schematic illustration showing anatomy of the larynx.

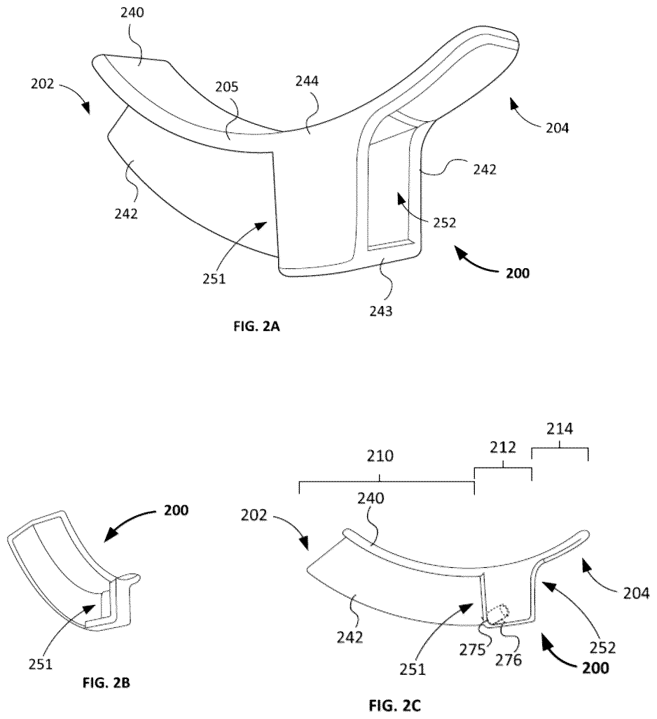

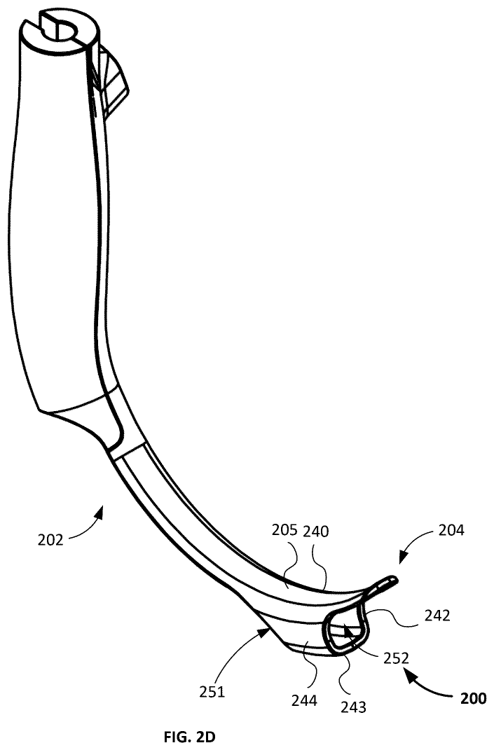

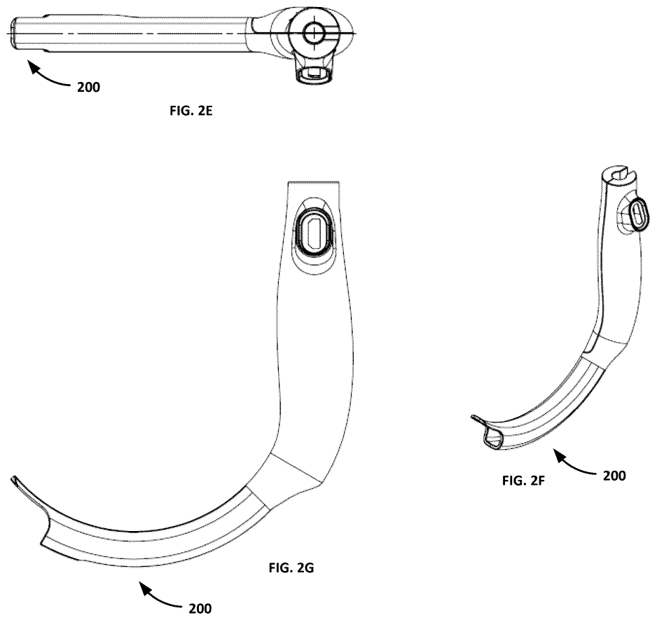

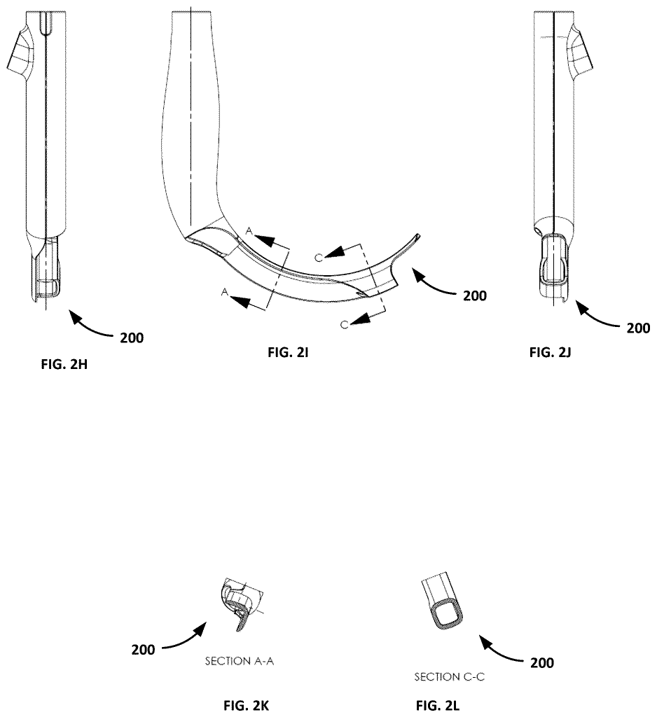

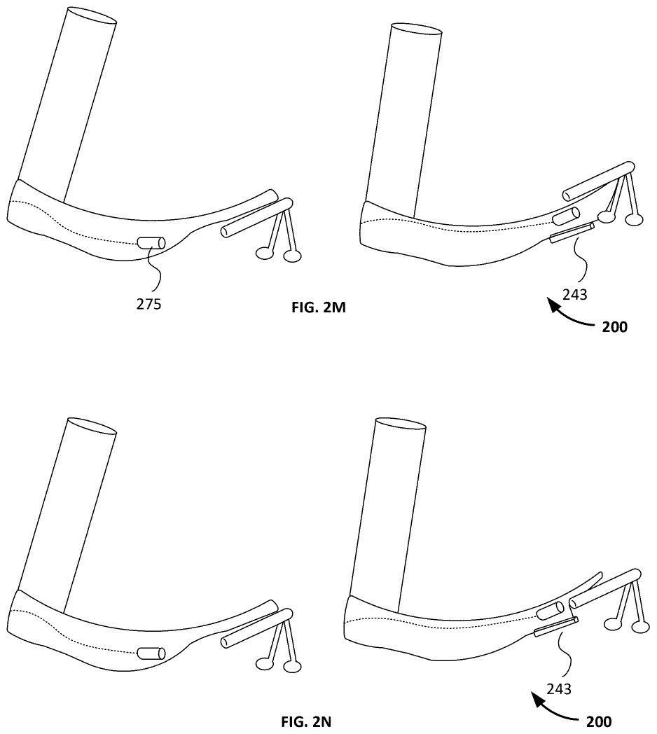

FIGS. 2A-2C are schematic perspective and side views of a blade of a laryngoscope according to one embodiment.

FIGS. 2D-2L are schematic views of another example of a laryngoscope according to one embodiment.

FIGS. 2M-2N are schematic illustrations showing the use of two laryngoscopes in connection with microlaryngeal surgery and a glottis/epiglottis operation, respectively.

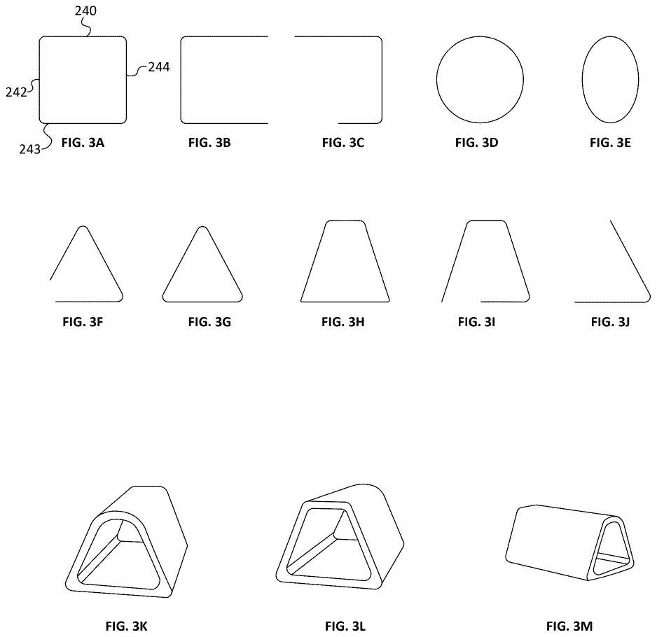

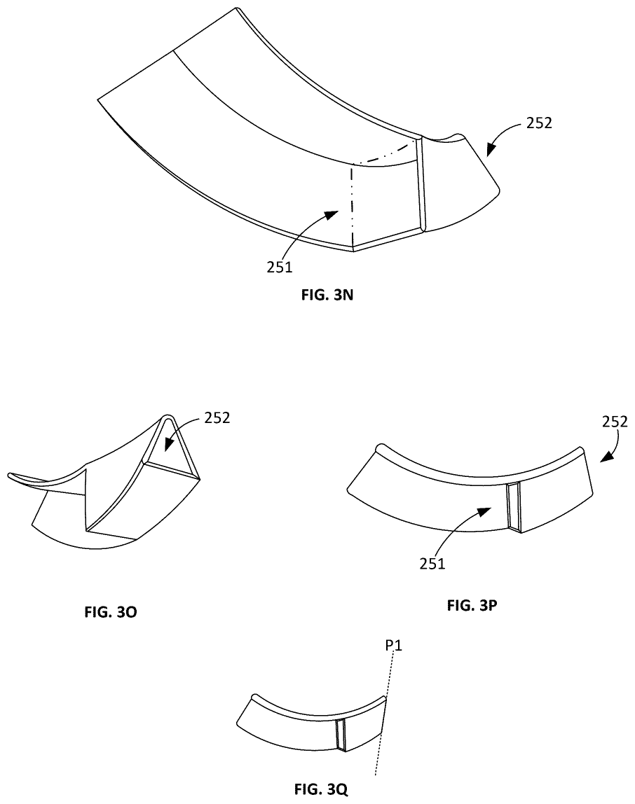

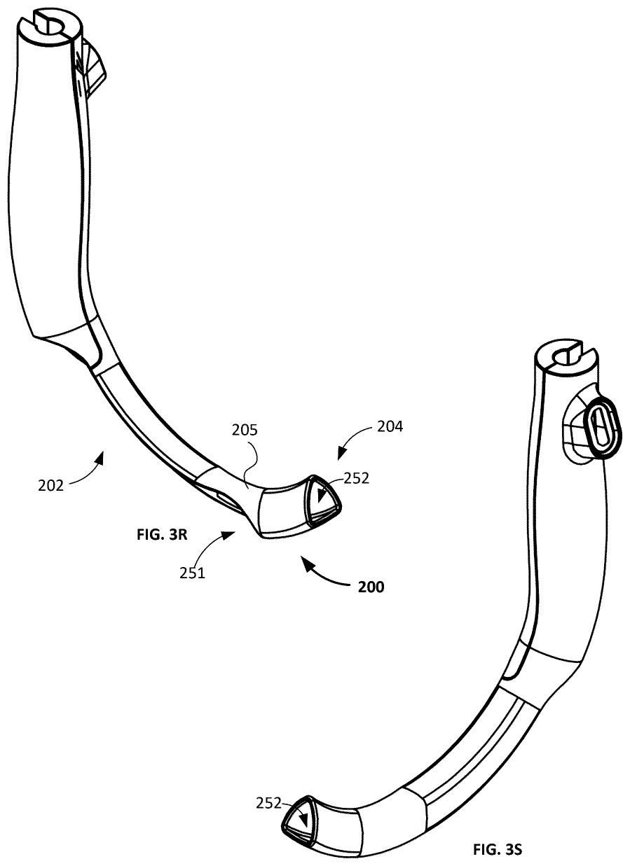

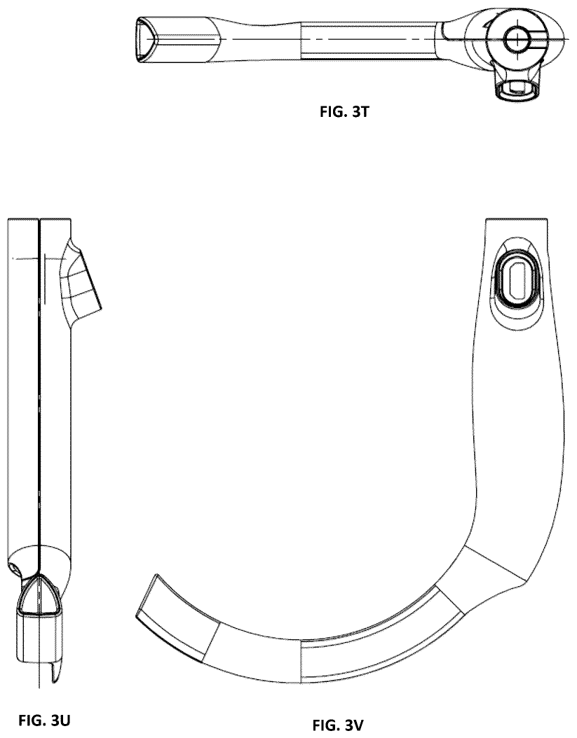

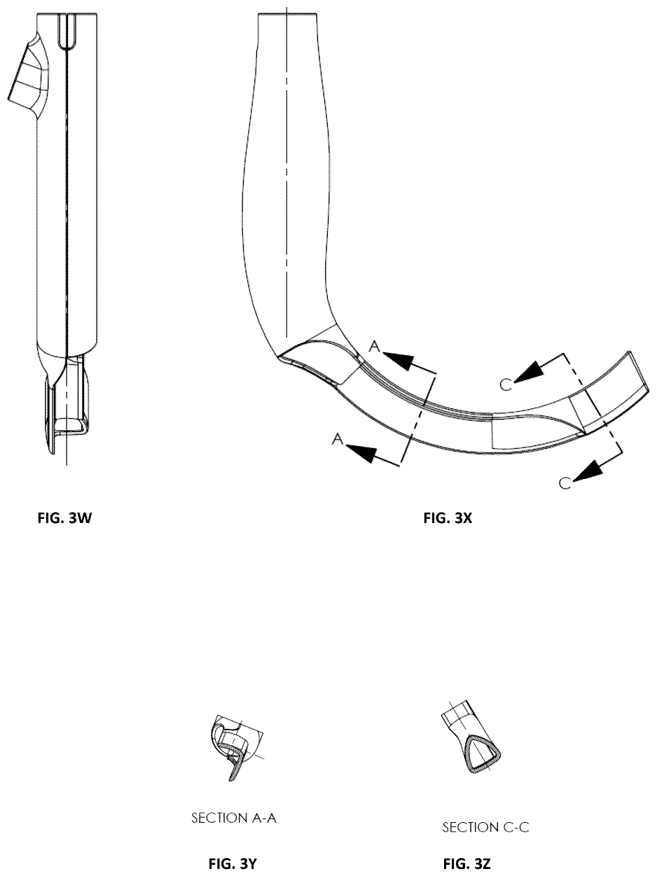

FIGS. 3A-3J illustrate various cross-sectional shapes for an intermediate section.

FIGS. 3K-3M illustrate an intermediate section with a transitioning cross-section.

FIGS. 3N-P illustrate an intermediate section that transitions from a rectangular-to-triangular cross-section.

FIG. 3Q is a schematic illustration of a blade having an oblique distal end.

FIGS. 3R-3Z are schematic views of another example of a laryngoscope according to one embodiment.

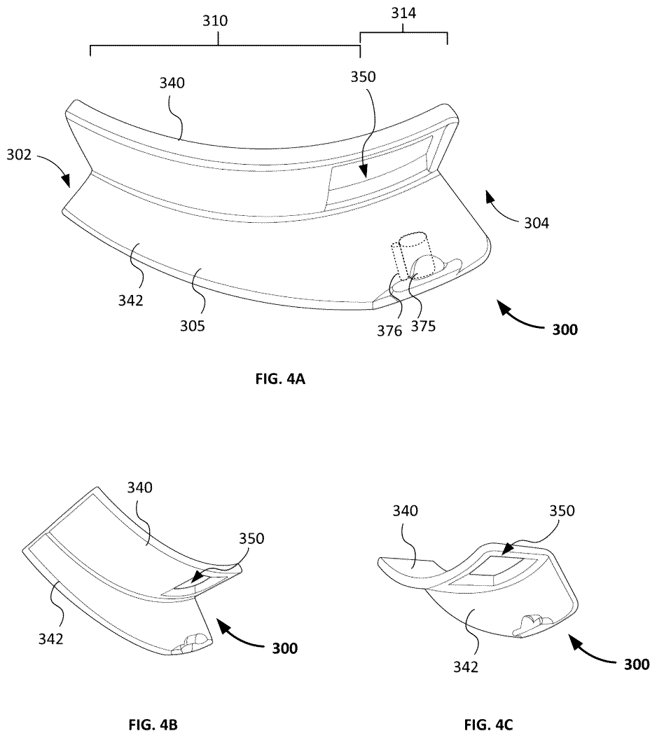

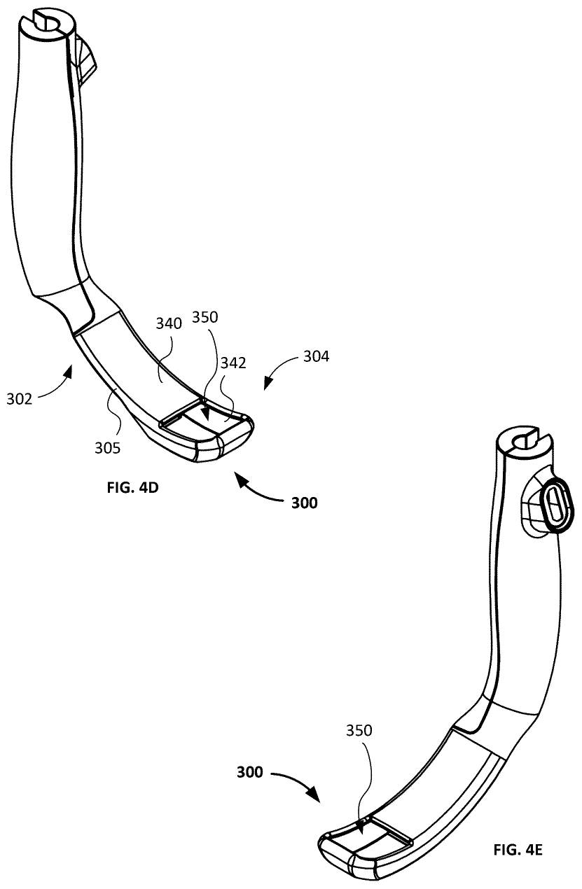

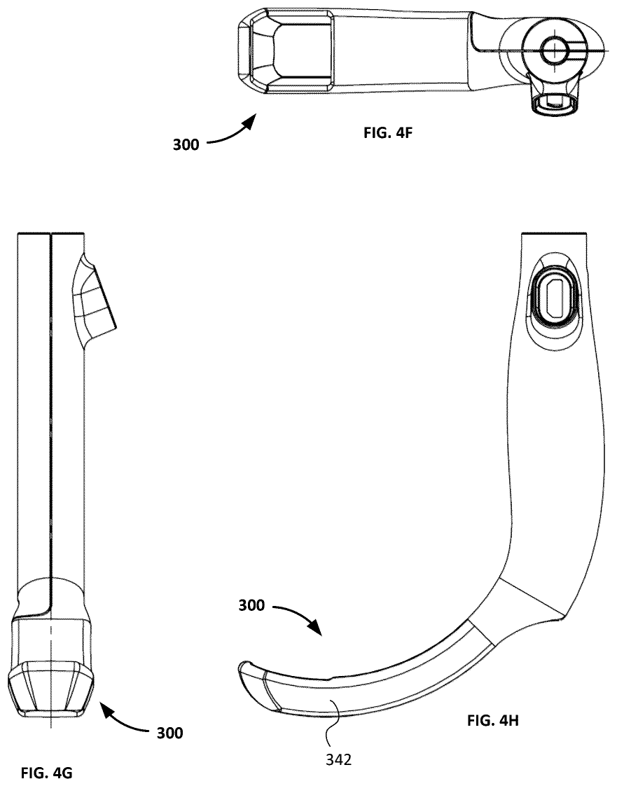

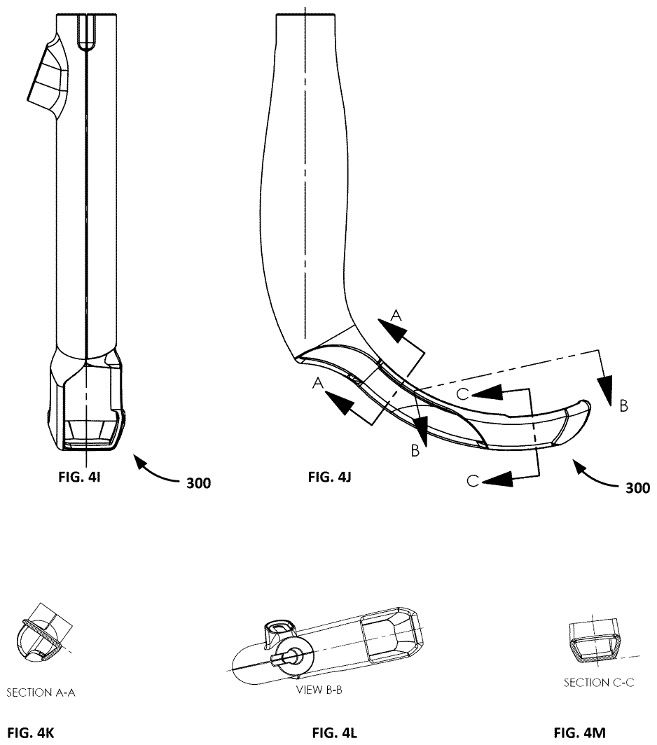

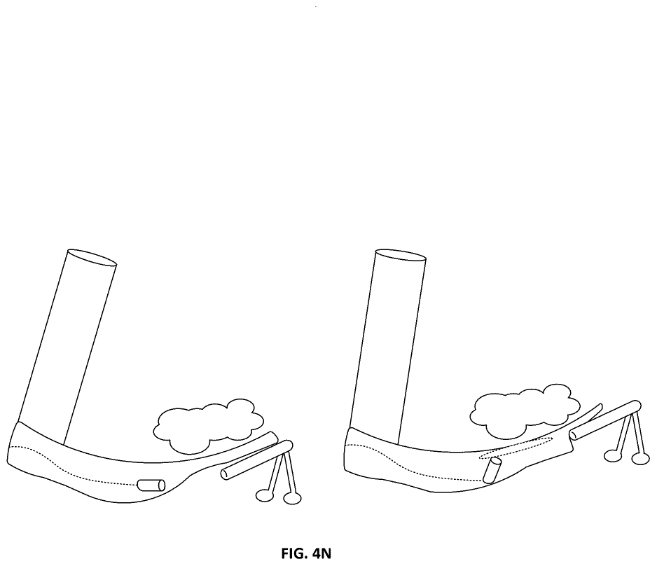

FIGS. 4A-4C are schematic perspective and side views of a blade of a laryngoscope for base of tongue operation.

FIGS. 4D-4M are schematic views of another example of a laryngoscope for base of tongue operation according to one embodiment.

FIG. 4N is a schematic illustration showing the use of a laryngoscope in connection with base of tongue surgery.

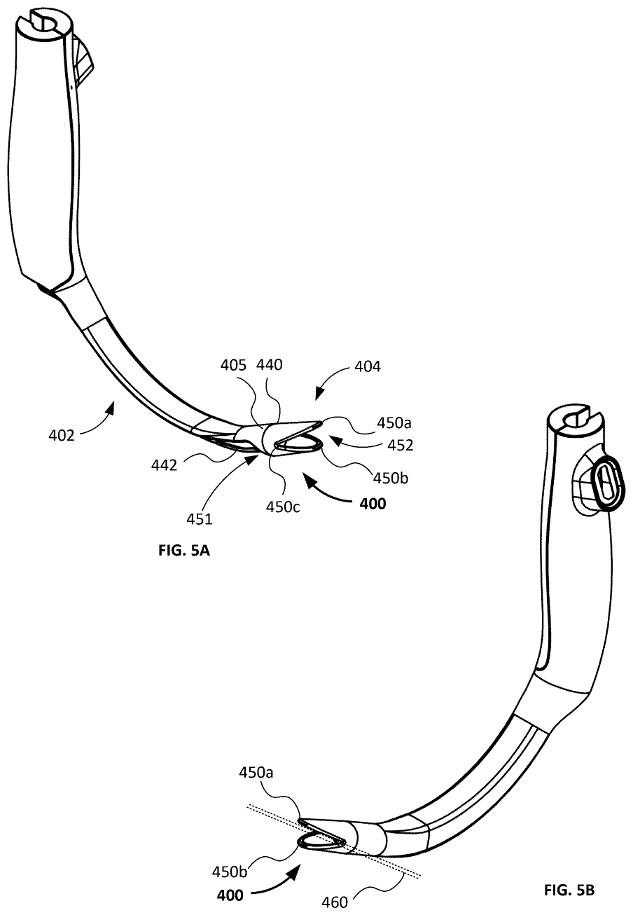

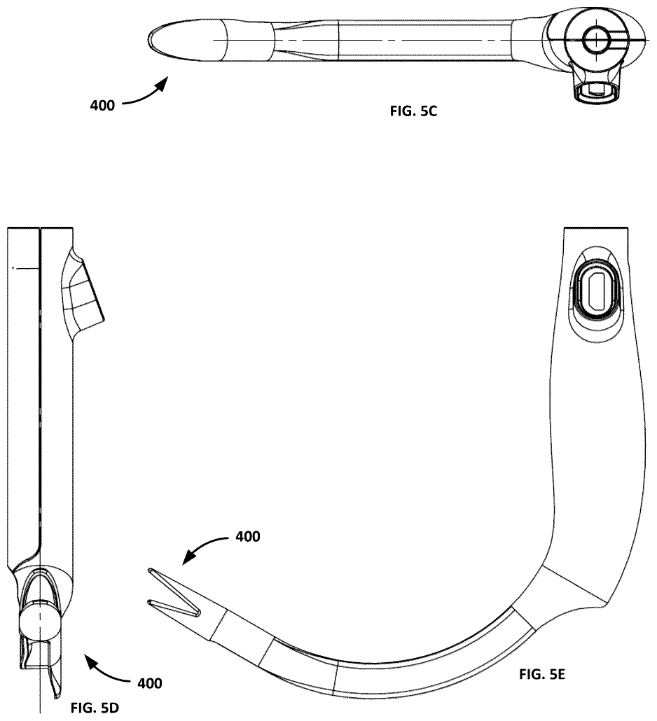

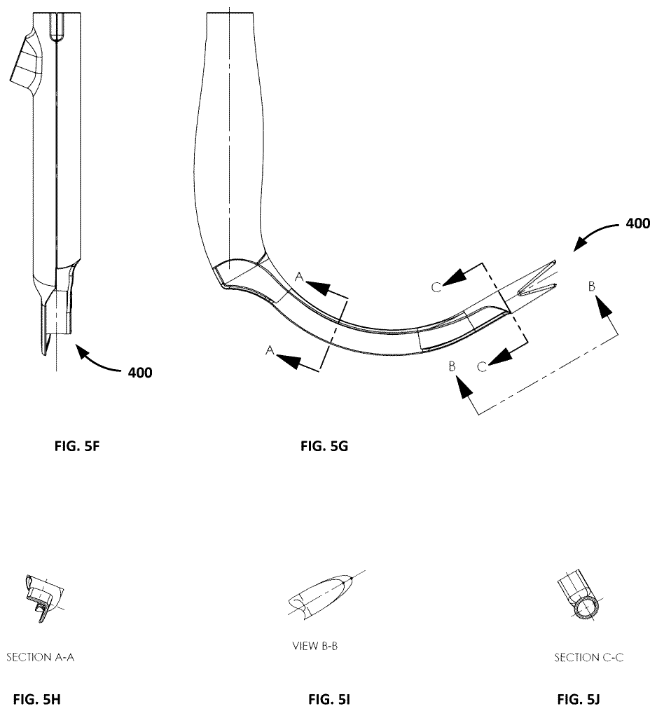

FIGS. 5A-5J are schematic views of another example of a laryngoscope for treating cricopharyngeal dysfunction according to one embodiment.

Various embodiments will now be described with reference to the appended drawings. It is to be appreciated that these drawings depict only some embodiments of the disclosure and are therefore not to be considered limiting of its scope.

### DETAILED DESCRIPTION

Despite the various improvements that have been made to laryngoscopes, conventional devices suffer from some shortcomings as discussed above.

Therefore, there is a need for further improvements to the devices and methods used for operative laryngoscopy. Among other advantages, the present disclosure may address one or more of these needs.

As used herein, the term “trailing” when used in connection with a laryngoscope or a component thereof refers to the end of the laryngoscope closer to the operator, while the term “leading” refers to the end of the laryngoscope or component farther from the operator. Likewise, the term “proximal,” refers to a direction relatively close to the user of that device or system when being used as intended, while the term “distal” refers to a direction relatively far from the user of the device. In other words, the leading end of a device or system is positioned distal to the trailing end of the delivery device or system, when being used as intended. As used herein, the terms “substantially,” “generally,” “approximately,” and “about” are intended to mean that slight deviations from absolute are included within the scope of the term so modified. Moreover, as used herein the term “upper” refers to the end of an element (e.g., the blade) closest to the handle and “lower” refers to an element farther from the handle.

Several embodiments of an operative laryngoscope are disclosed herein to allow for anatomic positioning which reduces complications via a partially open curved blade, and repositioning of video/light elements to allow for detailed microscopic view of the various pharyngeal and laryngeal anatomic sites (e.g., the base of tongue). The embodiments described herein may overcome common limitations and complications associated with a curved closed tube structure (e.g., custom curved instrumentation, single handed surgery, backhanded grip, and/or lack of stabilization for proper microsurgical technique).

In some embodiments, the shape, curvature, orientation and/or relative position of the various structure allow for repositioning of light and video elements, and the addition of a fulcrum for instrument stabilization so that a video laryngoscope can be used for surgical applications. The instant disclosure may also allow for application of standard single-use or reusable surgical instrumentation, as opposed to more expensive surgical systems utilizing flexible laparoscopic instruments or a surgical robot.

FIG. 1A illustrates a schematic perspective view of one embodiment of a laryngoscope 100 that extends from a proximal end 102 to a distal end 104. Laryngoscope 100 generally includes two main components: a handle 110 and a blade 120. The basic embodiments of laryngoscope 100 described herein may be used for associated operative visualization and instrument access to predetermined anatomic sites. For example, laryngoscope 100 may be directed to operation on the larynx, pharynx, and/or at the base of tongue. Any of the components disclosed herein may be formed of suitable materials such as plastic, metals, etc.

Laryngoscope 100 may be formed of a single solid, integrated structure with a handle 110 and a blade 120. Alternatively, the components of the laryngoscope may be separable, for example, along boundary 105 or interchangeable (e.g., the same handle 110 may be used with various types of blades, or blades of different sizes).

Handle 110 may be used for manual control during placement as the blade contacts and/or controls the position of the relevant anatomic structures of the tongue, cricopharyngeus muscle, pharynx, and/or larynx. Blade 120 may have a plurality of shapes and/or sizes, and may include an open paddle, triangular, crescent, open hoop, or other shaped cross-section. As shown in FIG. 1A, blade 120 includes a cross-section that is partially open (i.e., unbounded) and partially closed (i.e., bounded) and the details of this feature will be more fully described below. In some examples, laryngoscope 100 may contain cabling 131 to provide an integrated distal light and/or distal video components. Alternatively, these components may be removably couplable to the blade and/or handle. A proximal attachment for electronics related to the video and light systems may also be included. Laryngoscope 100 may contain a rigid component to serve as a fulcrum for balancing and controlling of surgical instruments. Optionally, laryngoscope 100 may include features for connection to a suspension apparatus having an adapter 132, an arm 133 and a tensioning knob 134, the arm having a securing element (e.g., a ring) for coupling to a table or other surface (not shown). Laryngoscope 100 may also include an integrated suction port at the distal end of the blade for continuous evacuation of surgical smoke plume. To better appreciate the potential environment for using laryngoscope 100, FIG. 1B is a schematic illustration of the anatomy of the larynx showing the epiglottis, the trachea and the vocal fold.

Turning to FIGS. 2A-C, a first embodiment of a portion of a blade 200 for use as part of a laryngoscope is shown. Blade 200 is configured and arranged for operation on the pharynx and/or larynx (to include the glottis, supraglottis, or subglottis) and/or microlaryngeal surgery. In this example, blade 200 includes a body 205 that extends between a proximal end 202 and a distal end 204 and generally includes three sections: a proximal section 210, an intermediate section 212 and an optional distal section 214 as best shown in FIG. 2C.

Beginning with proximal section 210, it will be seen that blade 200 may include body 205 having a curvilinear upper member 240 that extends from proximal end 202 to the distal end 204 across all three sections 210,212,214, or two sections 210,212 if the distal section is not present. A supporting first side member 242 may be coupled to upper member 240 and may extend therewith from proximal to distal ends with a matching lower curvature. In at least some examples, supporting side member 242 is disposed generally orthogonal to curvilinear upper member 240, although it will be understood that the angle between the upper member and the side member may be modified as desired. In this example, proximal section 210 includes only the upper member 240 and side member 242. Thus, it can be said that proximal section 210 includes an unbounded or open cross-section—that is, an instrument that is disposed below upper member 240 or next to side member 242 may freely move in two directions away from the upper member or the side member.

Conversely, intermediate section 212 may include a bounded shape formed of upper member 240, first side member 242, a lower member 243 opposite the upper member, and an opposing second side member 244. The “bounded” variation is used here by way of illustration, but it will be understood that the intermediate section 212 need not have a fully closed perimeter. Such examples will be described in greater detail below. In some examples, at least a portion of a lower member 243 is present opposite upper member 240, which may be used to support other instruments (e.g., to be used as a fulcrum), and this lower member 243 is coupled to the upper member 240 at one or both sides. The four members may form a desirable closed perimeter having a predetermined cross-section. In the example shown, the four members join each other at right angles and the intermediate section has a generally rectangular cross-section throughout the intermediate section. The closed intermediate section may define an inlet 251 having a first cross-section shape and an outlet 252 having a second cross-sectional shape.

In use, the operator may pass an instrument (e.g., one or more of injection needles, sharp or blunt probes, sharp or blunt dissectors, retractors, scissors, knives, forceps, suction, debriders, and/or surgical energy instrumentation such as laser, radio frequency, electrocautery, among others) or tool from inlet 251 toward outlet 252 to perform a surgical or therapeutic procedure on an anatomical structure distally disposed of the intermediate section. The use of an unbounded proximal section and a bounded (or semi-bounded) intermediate section simultaneously allow the operator to have a better line of sight through the proximal section, and also to use the intermediate section for supporting instruments. Specifically, the operator may rest, contact and/or use any portion of the intermediate section (e.g., the lower member) as a fulcrum for instruments during surgery for increased stability and ease of use.

FIGS. 2D-2L illustrate another example of a laryngoscope having one example of blade 200 including a body 205 that extends between a proximal end 202 and a distal end 204 and that has a curvilinear upper member 240, and a bounded shape formed of upper member 240, first side member 242, lower member 243 opposite the upper member, and an opposing second side member 244 similar to that previously described above with reference to FIGS. 2A-C. As shown in these drawings, only a portion of the blade is bounded while a proximal portion remains unbounded, which provides all of the benefits described above.

If available, optional distal section 214 may be used to lift or manipulate patient anatomy. Specifically, distal section 214 may include a protruding curved portion of upper member 240 configured and shaped to slide under the epiglottis toward the vocal cords for microlaryngeal surgery (See, FIG. 2M) or over the epiglottis for laryngeal procedures (FIG. 2N). In this example, distal section 214 includes only a portion of the upper member 240 and no other members. It will be appreciated, however, that the distal section 214 may include one or more portions of the side member 242, and may itself form a closed or bounded cross-section.

Optionally, a camera 275 or other image and/or video capturing element may be disposed within the proximal section 210 or the intermediate section 212 (e.g., within the bounded perimeter) and directed toward the vocal cords to assist in microlaryngeal surgery (FIG. 2C) or epiglottis/supraglottis operations (FIG. 2N). In at least some examples, the blade 200 includes a mating element 276 for fixing and coupling the camera 275 thereto. In at least some examples, mating element allows the camera to pivot thereabout to achieve a desirable line of sight.

FIGS. 2A-2L are provided by way of illustration. It will be understood that the closed cross-section at the inlet 251 and/or outlet 252 of the intermediate section 212 and the number of members or angles between the members may be modified based on the intended use. FIGS. 3A-J illustrate various cross-sectional shapes for the inlet 251 and/or outlet 252 of intermediate section 212, which may include for example, a rectangular cross-section (FIG. 3A), a circular cross-section (FIG. 3D), an oval cross-section (FIG. 3E), a triangular cross-section (FIG. 3G) and a trapezoidal cross-section (FIG. 3H). In addition to those, partially-bounded configurations are also possible where one or more sides or angles that would fully-bound the shape are missing (FIGS. 3B, 3C, 3F, 3I and 3J).

Additionally, the intermediate section 212 may begin with a first shaped cross-section at inlet 251 and extend to an opposing end at the outlet 252 having a second shaped cross-section. Any combination of the shapes described above may be used for the inlet 251 and/or outlet 252. For example, an intermediate section may smoothly transition from a rectangular cross-section to a triangular or trapezoidal cross-section (FIG. 3K-3M). FIGS. 3N-3P illustrate an intermediate section that includes a triangular cross-section near the outlet 252 and a rectangular cross-section adjacent the inlet 251. Another example of this particular configuration that includes a smooth transition from one shape to another from inlet to outlet is shown in FIGS. 3R-3Z, which illustrate a laryngoscope having a blade with a generally triangular outlet 252. Variations of these configurations are possible. In some examples, the distal end of a blade is oblique along an inclined plane P1 such that the top of the distal end extends distally to a farther position than the bottom of the distal end (See, FIG. 3Q). Although shown with the rectangular-to-triangular cross-sectional transition embodiment of FIG. 3Q, the oblique distal end may be combined with any of the other variations described herein.

Turning to FIGS. 4A-C, a second embodiment of a blade 300 for use as part of a laryngoscope is shown. Blade 300 is configured and arranged for operation on the base of the tongue. In this example, blade 300 includes body 305 that extends between a proximal end 302 and a distal end 304 and generally includes two sections: a proximal section 310 and a distal section 314. Blade 300 may include a curvilinear upper member 340 that extends from proximal end 302 to the distal end 304 across both sections 310,314. A supporting side member 342 may be coupled to upper member 340. In at least some examples, supporting side member 342 is disposed orthogonal to curvilinear upper member 340, although it will be understood that the angle between the upper member and the side member may be modified as desired.

In this example, upper member 340 includes a generally rectangular window 350 formed therein to allow the surgeon to view and operate on the base of the tongue. In at least some examples, window 350 is rectangular and defined near the edge of upper member 340. It will be understood that circular, oval, square, trapezoid or other-shaped windows are also possible. Optionally, a camera 375 and/or lighting elements 376 may be disposed below, and directed at, window 350 and coupled to side member 342. The positioning of camera 375 and light elements 376 inferiorly with an upward trajectory toward the window allows the operator to better visualize the anatomical structures through the open portion of the blade, namely the exposed area of the base of tongue. Another example of this particular configuration for use in a base of tongue operation is shown in FIGS. 4D-4L, which generally illustrate a laryngoscope having a blade with a window 350 near the edge of upper member 340 as previously described. This particular configuration may be used in operations (e.g., base of tongue operations) such as that shown in FIG. 4M.

Turning to FIGS. 5A-5J, another embodiment of a laryngoscope having a blade 400 is shown for use in treating cricopharyngeal dysfunction. Cricopharyngeal dysfunction or spasms occur when the cricopharyngeus muscle cannot relax, preventing food from entering the esophagus properly. In some cases, a Zenker's diverticulum may develop where a pouch is formed and enlarges and food may catch in this pouch or cause an obstruction. Treatments may include Botox injection, cricopharyngeal myotomy (where the cricopharyngeus muscle is cut to relieve tension), and/or elimination of a pharyngeal diverticular pouch. Blade 400 is configured and arranged for operation on the cricopharyngeus muscle region. In this example, blade 400 includes a body 405 that extends between a proximal end 402 and a distal end 404.

Similar to previous embodiments, body 405 may define an inlet 451 and an outlet 452. Proximal to inlet 451, body may include a single, unopposed side member 442 (i.e., a cutout is formed so that the side member does not face a second side member). The distal end of body 405 terminates in a curved upper jaw 450a and a curved lower jaw 450b with the outlet 452 being defined therebetween. In this example, upper jaw 450a and lower jaw 450b are coupled at two opposing junction 450c and form a bounded shape.

In use, the operator may pass an instrument (e.g., one or more of injection needles, sharp or blunt probes, sharp or blunt dissectors, retractors, scissors, knives, forceps, suction, debriders, and/or surgical energy instrumentation such as laser, radio frequency, electrocautery, among others) or tool from inlet 451 toward outlet 452 to perform a cricopharyngeal myotomy or treat a pharyngeal diverticulum. The use of an unbounded proximal section (i.e., the section with an unopposed side member) and a bounded (or semi-bounded) distal section (i.e., the jaws) simultaneously allow the operator to have a better line of sight through the proximal section, and also to use the intermediate section for supporting instruments. Additionally, the cricopharyngeus muscle may extend along travel path 460 (FIG. 5B) and may be stretched at junctions 450c between upper and lower jaws 450a,b to present it for cutting and/or treatment.

Thus, in some embodiments a surgical method comprises providing a laryngoscope including a handle, a blade coupled to the handle, the blade having a proximal section and an intermediate section, the proximal section having only an upper member and a first side member, and the intermediate section having a curved upper jaw and a curved lower jaw, the upper jaw and the lower jaw being joined at opposing junctions, placing a cricopharyngeus muscle between the curved upper jaw and the curved lower jaw, and passing a treatment device through the laryngoscope to treat the cricopharyngeus muscle.

It is to be understood that the embodiments described herein are merely illustrative of the principles and applications of the present disclosure. Other configuration, shapes and elements may be used to modify the blades and/or handles of the described laryngoscopes. For example, the cross-sectional shapes may be modified as desired or combined with one another to form various combinations of inlets, outlets and transitions therebetween. Additionally, windows may be formed in other portions of the blade. Moreover, the number, positioning and arrangement of members may be varied. Additionally, the shape, dimensions and arrangement of the body itself may also be varied as well as the curvatures described. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A laryngoscope comprising:
a handle; and
a blade coupled to the handle, the blade having a proximal section and an intermediate section, the proximal section having only an upper member and a first side member, and the intermediate section having the upper member, the first side member and a lower member.

2. The laryngoscope of claim 1, wherein the proximal section has an unbounded cross-sectional shape, and the intermediate section has a bounded cross-sectional shape.

3. The laryngoscope of claim 1, wherein the upper member is curvilinear, and the first side member is coupled to the upper member.

4. The laryngoscope of claim 1, wherein the intermediate section further includes a second side member, the first side member, the second side member, the upper member and the lower member defining a closed perimeter.

5. The laryngoscope of claim 4, wherein the closed perimeter is rectangular.

6. The laryngoscope of claim 1, wherein the intermediate section defines an inlet and an outlet, the inlet and the outlet having a same cross-sectional shape.

7. The laryngoscope of claim 1, wherein the intermediate section defines an inlet and an outlet, the inlet and the outlet having different cross-sectional shapes.

8. The laryngoscope of claim 1, wherein the intermediate section defines an inlet and an outlet, the outlet having a triangular cross-sectional shape.

9. The laryngoscope of claim 1, wherein the intermediate section defines an inlet and an outlet, the outlet having a rectangular cross-sectional shape.

10. The laryngoscope of claim 1, wherein the intermediate section defines an inlet and an outlet, the outlet having a trapezoidal cross-sectional shape.

11. The laryngoscope of claim 1, wherein the intermediate section defines an inlet and an outlet, and the intermediate section defines a series of cross-sections that transition from the inlet to the outlet.

12. The laryngoscope of claim 1, further comprising a distal section having an unbounded shape.

13. The laryngoscope of claim 12, wherein the distal section includes only the upper member.

14. A laryngoscope comprising:
a handle; and
a blade coupled to the handle, the blade having a proximal section and an intermediate section, the proximal section having an upper member and a first side member, and the intermediate section having the upper member and the first side member and defining a window formed in the upper member.

15. The laryngoscope of claim 14, wherein the window is rectangular.

16. The laryngoscope of claim 14, wherein the window is rounded.

17. A surgical method, comprising:
providing a laryngoscope including a handle, a blade coupled to the handle, the blade having a proximal section and an intermediate section, the proximal section having only an upper member and a first side member, and the intermediate section having the upper member, the first side member and a lower member;
introducing the blade of the laryngoscope to a patient's throat; and
passing an instrument through the proximal section of the blade to the intermediate section of the blade.

18. The surgical method of claim 17, further comprising the step of using a portion of the intermediate section of the blade to support the instrument.

19. The surgical method of claim 18, further comprising the step of using the lower member of the blade to support the instrument.

20. The surgical method of claim 18, further comprising the step of using the lower member as a fulcrum.
